(19) United States
(12) Patent Application Publication
(10) Pub. No.: US 2023/0392113 A1
(43) Pub. Date: Dec. 7, 2023

(54) CELLULAR REGENERATIVE THERAPEUTICS FOR ENHANCEMENT/RESTORATION OF ENDOMETRIAL FUNCTION

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Niceville, FL (US)

(73) Assignee: CREATIVE MEDICAL TECHNOLOGIES, INC., Phoenix, AZ (US)

(21) Appl. No.: 18/331,029

(22) Filed: Jun. 7, 2023

Related U.S. Application Data
(60) Provisional application No. 63/349,976, filed on Jun. 7, 2022.

(51) Int. Cl. C12N 5/0735

(52) U.S. Cl. CPC C12N 5/0606; C12N 2500/30; C12N 2506/45

(57) ABSTRACT

Disclosed are methods, compositions of matter and protocols useful for restoring/enhancing endometrial function by administration of regenerative cells. In one embodiment cells capable of decreasing fibrosis, stimulation of angiogenesis, and augmenting hormone responsiveness are administered systemically or locally. In some embodiments cells utilized are autologous or allogeneic mesenchymal stem cells.

### CROSS-REFERENCE TO RELATED APPLICATIONS

This application claims priority to U.S. Provisional Application No. 63/349,976 titled “Cellular Regenerative Therapeutics for Enhancement/Restoration of Endometrial Function” filed Jun. 7, 2022, which is hereby incorporated by reference herein in its entirety.

### FIELD OF THE INVENTION

The invention relates to the use of cellular therapeutics for the regenerative treatment of the endometrium.

### BACKGROUND OF THE INVENTION

During the ages when women are capable of reproduction, there are two layers of the endometrium that can be distinguished: (i) the functional layer adjacent to the uterine cavity, and (ii) the basal layer, adjacent to the myometrium and below the functional layer. The functional layer is built up after the end of menstruation during the first part of the previous menstrual cycle. Proliferation is induced by estrogen (follicular phase of menstrual cycle), and later changes in this layer are produced by progesterone from the corpus luteum (luteal phase). It is adapted to provide an optimum environment for the implantation and growth of the embryo.

It is widely recognized that the layer that is functional is completely shed during menstruation. In contrast, the basal layer is not shed at any time during the menstrual cycle. Regeneration of the human endometrium under systemic ovarian steroids changes in each menstrual cycle is essential for the preparation of this organ for its main function, i.e., the development of the endometrial window of implantation to host the implanting blastocyst, allowing pregnancy to occur.

Replenishment of all cellular compartments of the endometrial functional layer with each menstrual cycle is essential for normal reproductive function.

It is known that Asherman's Syndrome (AS) is a condition in which there is a destruction of the endometrium caused by repeated or aggressive curettages and/or endometritis. It produces an obliteration of the uterine cavity with intrauterine adhesions and absence of functional endometrium in many areas. Women with this disease as well with atrophic endometrium (<4 mm) often struggle with infertility, menstrual irregularities including amenorrhea, hypomenorrhea, and recurrent pregnancy losses. Currently no specific treatment for these endometrial pathologies exist. Thus, there remains a need to develop safe and effective therapies to treat these pathologies.

The invention teaches that administration of various regenerative cells such as mesenchymal stem cells are capable of repairing damaged endometrial tissue. This includes preservation of tissue through inhibition of apoptosis, stimulation of angiogenesis, and regression of fibrosis or scar tissue formation.

### SUMMARY

Preferred embodiments are drawn to methods of preventing and/or treating endometrial atrophy, comprising the steps of: a) selecting a patient in need of treatment; b) administering an effective number of regenerative cells into the subject in need of treatment; c) assessing effect of regenerative cell injecting/infusing and adjusting concentration and frequency based on response.

Preferred methods include embodiments wherein said regenerative cells are administered into the ovarian and/or uterine artery.

Preferred methods include embodiments wherein said regenerative cells are mesenchymal stem cell are derived from tissue comprising a group selected from: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) cord or placental tissue.

Preferred methods include embodiments wherein said mesenchymal stem cells express a marker or plurality of markers selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

Preferred methods include embodiments wherein said mesenchymal stem cells do not express substantial levels of HLA-DR, CD117, and CD45.

Preferred methods include embodiments wherein said mesenchymal stem cells are generated from a pluripotent stem cell.

Preferred methods include embodiments wherein said pluripotent stem cell is selected from a group comprising of: a) an embryonic stem cell; b) an inducible pluripotent stem cell; c) a parthenogenic stem cell; and d) a somatic cell nuclear transfer derived stem cell.

Preferred methods include embodiments wherein said embryonic stem cell population expresses genes selected from a group comprising of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

Preferred methods include embodiments wherein said inducible pluripotent stem cell possesses markers selected from a group comprising of: CD10, CD13, CD44, CD73, CD90, PDGFr-alpha, PD-L2, and HLA-A,B,C and possesses ability to undergo at least 40 doublings in culture, while maintaining a normal karyotype upon passaging.

10. The method of claim 7, wherein said parthenogenic stem cells wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.

Preferred methods include embodiments wherein said somatic cell nuclear transfer derived stem cells possess a phenotype negative for SSEA-1 and positive for SSEA-3, SSEA-4, TRA-1-60, TRA-1-81, and alkaline phosphatase.

Preferred methods include embodiments wherein said mesenchymal stem cells are differentiated from a pluripotent stem cell source through culture in the presence of an inhibitor of the SMAD-2/3 pathway.

Preferred methods include embodiments wherein said mesenchymal stem cells are differentiated from a pluripotent stem cell source through culture in the presence of an inhibitor nucleic acid targeting the SMAD-2/3 pathway.

Preferred methods include embodiments wherein said nucleic acid inhibitor is selected from a group comprising of: a) an antisense oligonucleotide; b) a hairpin loop short interfering RNA; c) a chemically synthesized short interfering RNA molecule; and d) a hammerhead ribozyme.

Preferred methods include embodiments wherein said inhibitor of the SMAD-2/3 pathway is a small molecule inhibitor.

Preferred methods include embodiments wherein said small molecule inhibitor is SB-431542.

Preferred methods include embodiments wherein a selection process is used to enrich for mesenchymal stem cells differentiated from said pluripotent stem cell population.

Preferred methods include embodiments wherein said enrichment method comprises of positively selecting for cells expressing a marker associated with mesenchymal stem cells.

Preferred methods include embodiments wherein said marker of mesenchymal stem cells is selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

Preferred methods include embodiments wherein the subject is known to have Asherman's syndrome.

Preferred methods include embodiments wherein the subject has endometrial atrophy that is resistant to hormonal or other treatments.

Preferred methods include embodiments wherein the subject has had one or more prior embryo implantation failures.

Preferred methods include embodiments wherein the regenerative cells are prepared by administering to the subject an agent to mobilize regenerative cells from bone marrow into peripheral blood of the subject; and isolating said regenerative cells from peripheral blood of the subject.

Preferred methods include embodiments wherein the agent to mobilize regenerative cells is granulocyte colony-stimulating factor (G-CSF).

Preferred methods include embodiments wherein the agent to mobilize regenerative cells is granulocyte monocyte colony-stimulating factor (GM-CSF).

Preferred methods include embodiments wherein the agent to mobilize regenerative cells is Leukemia Inhibiting Factor (LIF).

Preferred methods include embodiments wherein the agent to mobilize regenerative cells is HGF-1.

Preferred methods include embodiments wherein the agent to mobilize regenerative cells is FGF-1.

Preferred methods include embodiments wherein the agent to mobilize regenerative cells is FGF-2.

Preferred methods include embodiments wherein the agent to mobilize regenerative cells is AMD-3100.

Preferred methods include embodiments wherein the agent to mobilize regenerative cells is M=CSF.

Preferred methods include embodiments wherein the agent to mobilize regenerative cells is ozone therapy.

Preferred methods include embodiments wherein the agent to mobilize regenerative cells is IL-2.

Preferred methods include embodiments wherein the agent to mobilize regenerative cells is FLT-3 ligand.

Preferred methods include embodiments wherein the agent to mobilize regenerative cells is TNF alpha.

Preferred methods include embodiments wherein the agent to mobilize regenerative cells is hCG.

Preferred methods include embodiments wherein the agent to mobilize regenerative cells is hyperbaric oxygen.

Preferred methods include embodiments wherein the agent to mobilize regenerative cells is BDNF.

Preferred methods include embodiments wherein the agent to mobilize regenerative cells is NGF-1.

Preferred methods include embodiments wherein the agent to mobilize regenerative cells is VEGF.

Preferred methods include embodiments wherein the regenerative cells are isolated from peripheral circulation of the subject by apheresis using an antibody that has selective affinity to said regenerative cells.

Preferred methods include embodiments wherein said regenerative cells are isolated with an antibody capable of binding cells expressing CD31.

Preferred methods include embodiments wherein said regenerative cells are isolated with an antibody capable of binding cells expressing CD33.

Preferred methods include embodiments wherein said regenerative cells are isolated with an antibody capable of binding cells expressing CD34.

Preferred methods include embodiments wherein said regenerative cells are isolated with an antibody capable of binding cells expressing CD133.

Preferred methods include embodiments wherein said regenerative cells are isolated with an antibody capable of binding cells expressing CD34.

Preferred methods include embodiments wherein said regenerative cells are isolated with an antibody capable of binding cells expressing CD31 and CD34.

Preferred methods include embodiments wherein said regenerative cells are isolated with an antibody capable of binding cells expressing CD31 and CD33.

Preferred methods include embodiments wherein said regenerative cells are isolated with an antibody capable of binding cells expressing CD31 and VEGF receptor.

Preferred methods include embodiments wherein said regenerative cells are isolated with an antibody capable of binding cells expressing CD31 and HGF-1 receptor.

Preferred methods include embodiments wherein said regenerative cells are isolated with an antibody capable of binding cells expressing CD31 and stem cell factor.

Preferred methods include embodiments wherein said regenerative cells are isolated with an antibody capable of binding cells expressing TLR-4 and CD34.

Preferred methods include embodiments wherein said regenerative cells are isolated with an antibody capable of binding cells expressing TLR-4 and CD33.

Preferred methods include embodiments wherein said regenerative cells are isolated with an antibody capable of binding cells expressing TLR-4 and VEGF receptor.

Preferred methods include embodiments wherein said regenerative cells are isolated with an antibody capable of binding cells expressing TLR-4 and HGF-1 receptor.

Preferred methods include embodiments wherein said regenerative cells are isolated with an antibody capable of binding cells expressing TLR-4 and stem cell factor.

Preferred methods include embodiments wherein said regenerative cells are isolated with an antibody capable of binding cells expressing TLR-4 and CD90.

Preferred methods include embodiments wherein said regenerative cells are isolated with an antibody capable of binding cells expressing TLR-4 and CD13.

Preferred methods include embodiments wherein said regenerative cells are isolated with an antibody capable of binding cells expressing TLR-4 and CD29.

Preferred methods include embodiments wherein said regenerative cells are isolated with an antibody capable of binding cells expressing TLR-4 and CD44.

Preferred methods include embodiments wherein said regenerative cells are isolated with an antibody capable of binding cells expressing TLR-4 and CD71.

Preferred methods include embodiments wherein said regenerative cells are isolated with an antibody capable of binding cells expressing TLR-4 and CD73.

Preferred methods include embodiments wherein said regenerative cells are isolated with an antibody capable of binding cells expressing TLR-4 and CD105.

Preferred methods include embodiments wherein said regenerative cells are isolated with an antibody capable of binding cells expressing TLR-4 and CD166.

Preferred methods include embodiments wherein said regenerative cells are isolated with an antibody capable of binding cells expressing TLR-4 and STRO-1.

Preferred methods include embodiments wherein said regenerative cells are isolated with an antibody capable of binding cells expressing TLR-4 and STRO-4.

Preferred methods include embodiments wherein said regenerative cells are isolated with an antibody capable of binding cells expressing TLR-4 and TNF receptor p55.

Preferred methods include embodiments wherein said regenerative cells are isolated with an antibody capable of binding cells expressing TLR-4 and TNF receptor p75.

Preferred methods include embodiments wherein said regenerative cells are isolated with an antibody capable of binding cells expressing TLR-4 and CD227.

Preferred methods include embodiments wherein said regenerative cells are isolated with an antibody capable of binding cells expressing c-kit and CD34.

Preferred methods include embodiments wherein said regenerative cells are isolated with an antibody capable of binding cells expressing c-kit and CD33.

Preferred methods include embodiments wherein said regenerative cells are isolated with an antibody capable of binding cells expressing c-kit and VEGF receptor.

Preferred methods include embodiments wherein said regenerative cells are isolated with an antibody capable of binding cells expressing c-kit and HGF-1 receptor.

Preferred methods include embodiments wherein said regenerative cells are isolated with an antibody capable of binding cells expressing c-kit and stem cell factor.

Preferred methods include embodiments wherein said regenerative cells are isolated with an antibody capable of binding cells expressing c-kit and CD90.

Preferred methods include embodiments wherein said regenerative cells are isolated with an antibody capable of binding cells expressing c-kit and CD13.

Preferred methods include embodiments wherein said regenerative cells are isolated with an antibody capable of binding cells expressing c-kit and CD29.

Preferred methods include embodiments wherein said regenerative cells are isolated with an antibody capable of binding cells expressing c-kit and CD44.

Preferred methods include embodiments wherein said regenerative cells are isolated with an antibody capable of binding cells expressing c-kit and CD71.

Preferred methods include embodiments wherein said regenerative cells are isolated with an antibody capable of binding cells expressing c-kit and CD73.

Preferred methods include embodiments wherein said regenerative cells are isolated with an antibody capable of binding cells expressing c-kit and CD105.

Preferred methods include embodiments wherein said regenerative cells are isolated with an antibody capable of binding cells expressing c-kit and CD166.

Preferred methods include embodiments wherein said regenerative cells are isolated with an antibody capable of binding cells expressing c-kit and STRO-1.

Preferred methods include embodiments wherein said regenerative cells are isolated with an antibody capable of binding cells expressing c-kit and STRO-4.

Preferred methods include embodiments wherein said regenerative cells are isolated with an antibody capable of binding cells expressing c-kit and TNF receptor p55.

Preferred methods include embodiments wherein said regenerative cells are isolated with an antibody capable of binding cells expressing c-kit and TNF receptor p75.

Preferred methods include embodiments wherein said regenerative cells are isolated with an antibody capable of binding cells expressing c-kit and CD227.

Preferred methods include embodiments wherein said regenerative cells are isolated with an antibody capable of binding cells expressing OCT-4 and CD34.

Preferred methods include embodiments wherein said regenerative cells are isolated with an antibody capable of binding cells expressing OCT-4 and CD33.

Preferred methods include embodiments wherein said regenerative cells are isolated with an antibody capable of binding cells expressing OCT-4 and VEGF receptor.

Preferred methods include embodiments wherein said regenerative cells are isolated with an antibody capable of binding cells expressing OCT-4 and HGF-1 receptor.

Preferred methods include embodiments wherein said regenerative cells are isolated with an antibody capable of binding cells expressing OCT-4 and stem cell factor.

Preferred methods include embodiments wherein said regenerative cells are isolated with an antibody capable of binding cells expressing OCT-4 and CD90.

Preferred methods include embodiments wherein said regenerative cells are isolated with an antibody capable of binding cells expressing OCT-4 and CD13.

Preferred methods include embodiments wherein said regenerative cells are isolated with an antibody capable of binding cells expressing OCT-4 and CD29.

Preferred methods include embodiments wherein said regenerative cells are isolated with an antibody capable of binding cells expressing OCT-4 and CD44.

Preferred methods include embodiments wherein said regenerative cells are isolated with an antibody capable of binding cells expressing OCT-4 and CD71.

Preferred methods include embodiments wherein said regenerative cells are isolated with an antibody capable of binding cells expressing OCT-4 and CD73.

Preferred embodiments include methods of preventing or treating endometrial atrophy comprising of administering regenerative cells combined with endometrial stimulating factors.

Preferred methods include embodiments wherein said regenerative cells are mesenchymal stem cells.

Preferred methods include embodiments wherein said mesenchymal stem cells are naturally occurring mesenchymal stem cells.

Preferred methods include embodiments wherein said mesenchymal stem cells are generated in vitro.

Preferred methods include embodiments wherein said naturally occurring mesenchymal stem cells are tissue derived.

Preferred methods include embodiments wherein said naturally occurring mesenchymal stem cells are derived from a bodily fluid.

Preferred methods include embodiments wherein said tissue derived mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

Preferred methods include embodiments wherein said tissue derived mesenchymal stem cells are isolated from tissues containing cells selected from a group of cells comprising of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

Preferred methods include embodiments wherein said mesenchymal stem cells are plastic adherent.

Preferred methods include embodiments wherein said mesenchymal stem cells express a marker selected from a group comprising of: a) CD73; b) CD90; and c) CD105.

Preferred methods include embodiments wherein said mesenchymal stem cells lack expression of a marker selected from a group comprising of: a) CD14; b) CD45; and c) CD34.

Preferred methods include embodiments wherein said mesenchymal stem cells from umbilical cord tissue express markers selected from a group comprising of; a) oxidized low density lipoprotein receptor 1, b) chemokine receptor ligand 3; and c) granulocyte chemotactic protein.

Preferred methods include embodiments wherein said mesenchymal stem cells from umbilical cord tissue do not express markers selected from a group comprising of: a) CD117; b) CD31; c) CD34; and CD45.

Preferred methods include embodiments wherein said mesenchymal stem cells from umbilical cord tissue express, relative to a human fibroblast, increased levels of interleukin 8 and reticulon 1.

Preferred methods include embodiments wherein said mesenchymal stem cells from umbilical cord tissue have the potential to differentiate into cells of at least a skeletal muscle, vascular smooth muscle, pericyte or vascular endothelium phenotype.

Preferred methods include embodiments wherein said mesenchymal stem cells from umbilical cord tissue express markers selected from a group comprising of: a) CD10; b) CD13; c) CD44; d) CD73; and e) CD90.

Preferred methods include embodiments wherein said umbilical cord tissue mesenchymal stem cell is an isolated umbilical cord tissue cell isolated from umbilical cord tissue substantially free of blood that is capable of self-renewal and expansion in culture,

Preferred methods include embodiments wherein said umbilical cord tissue mesenchymal stem cells has the potential to differentiate into cells of other phenotypes.

Preferred methods include embodiments wherein said other phenotypes comprise: a) osteocytic; b) adipogenic; and c) chondrogenic differentiation.

Preferred methods include embodiments wherein said cord tissue derived mesenchymal stem cells can undergo at least 20 doublings in culture.

Preferred methods include embodiments wherein said cord tissue derived mesenchymal stem cell maintains a normal karyotype upon passaging.

Preferred methods include embodiments wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C.

Preferred methods include embodiments wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR, DP, DQ.

Preferred methods include embodiments wherein said umbilical cord tissue-derived cell secretes factors selected from a group comprising of: a) MCP-1; b) MIPlbeta; c) IL-6; d) IL-8; e) GCP-2; 0 HGF; g) KGF; h) FGF; i) HB-EGF; j) BDNF; k) TPO; l) RANTES; and m) TIMP1.

Preferred methods include embodiments wherein said umbilical cord tissue derived cells express markers selected from a group comprising of: a) TRA1-60; b) TRA1-81; c) SSEA3; d) SSEA4; and e) NANOG.

Preferred methods include embodiments wherein said umbilical cord tissue-derived cells are positive for alkaline phosphatase staining.

Preferred methods include embodiments wherein said umbilical cord tissue-derived cells are capable of differentiating into one or more lineages selected from a group comprising of; a) ectoderm; b) mesoderm, and; c) endoderm.

Preferred methods include embodiments wherein said bone marrow derived mesenchymal stem cells possess markers selected from a group comprising of: a) CD73; b) CD90; and c) CD105.

Preferred methods include embodiments wherein said bone marrow derived mesenchymal stem cells possess markers selected from a group comprising of: a) LFA-3; b) ICAM-1; c) PECAM-1; d) P-selectin; e) L-selectin; f) CD49b/CD29; g) CD49c/CD29; h) CD49d/CD29; i) CD29; j) CD18; k) CD61; l) 6-19; m) thrombomodulin; n) telomerase; o) CD10; p) CD13; and q) integrin beta.

Preferred methods include embodiments wherein said bone marrow derived mesenchymal stem cell is a mesenchymal stem cell progenitor cell.

Preferred methods include embodiments wherein said mesenchymal progenitor cells are a population of bone marrow mesenchymal stem cells enriched for cells containing STRO-1.

Preferred methods include embodiments wherein said mesenchymal progenitor cells express both STRO-1 and VCAM-1.

Preferred methods include embodiments wherein said STRO-1 expressing cells are negative for at least one marker selected from the group consisting of: a) CBFA-1; b) collagen type II; c) PPAR.gamma2; d) osteopontin; e) osteocalcin; f) parathyroid hormone receptor; g) leptin; h) H-ALBP; i) aggrecan; j) Ki67, and k) glycophorin A.

Preferred methods include embodiments wherein said bone marrow mesenchymal stem cells lack expression of CD14, CD34, and CD45.

Preferred methods include embodiments wherein said STRO-1 expressing cells are positive for a marker selected from a group comprising of: a) VACM-1; b) TKY-1; c) CD146 and; d) STRO-2.

Preferred methods include embodiments wherein said bone marrow mesenchymal stem cell express markers selected from a group comprising of: a) CD13; b) CD34; c) CD56 and; d) CD117.

Preferred methods include embodiments wherein said bone marrow mesenchymal stem cells do not express CD10.

Preferred methods include embodiments wherein said bone marrow mesenchymal stem cells do not express CD2, CD5, CD14, CD19, CD33, CD45, and DRII.

Preferred methods include embodiments wherein said bone marrow mesenchymal stem cells express CD13, CD34, CD56, CD90, CD117 and nestin, and which do not express CD2, CD3, CD10, CD14, CD16, CD31, CD33, CD45 and CD64.

Preferred methods include embodiments wherein said skeletal muscle stem cells express markers selected from a group comprising of: a) CD13; b) CD34; c) CD56 and; d) CD117.

Preferred methods include embodiments wherein said skeletal muscle mesenchymal stem cells do not express CD10.

Preferred methods include embodiments wherein said skeletal muscle mesenchymal stem cells do not express CD2, CD5, CD14, CD19, CD33, CD45, and DRII.

Preferred methods include embodiments wherein said bone marrow mesenchymal stem cells express CD13,CD34, CD56, CD90, CD117 and nestin, and which do not express CD2, CD3, CD10, CD14, CD16, CD31, CD33, CD45 and CD64.

Preferred methods include embodiments wherein said subepithelial umbilical cord derived mesenchymal stem cells possess markers selected from a group comprising of; a) CD29; b) CD73; c) CD90; d) CD166; e) SSEA4; f) CD9; g) CD44; h) CD146; and i) CD105.

Preferred methods include embodiments wherein said subepithelial umbilical cord derived mesenchymal stem cells do not express markers selected from a group comprising of; a) CD45; b) CD34; c) CD14; d) CD79; e) CD106; f) CD86; g) CD80; h) CD19; i) CD117; j) Stro-1 and k) HLA-DR.

Preferred methods include embodiments wherein said subepithelial umbilical cord derived mesenchymal stem cells express CD29, CD73, CD90, CD166, SSEA4, CD9, CD44, CD146, and CD105.

Preferred methods include embodiments wherein said subepithelial umbilical cord derived mesenchymal stem cells do not express CD45, CD34, CD14, CD79, CD106, CD86, CD80, CD19, CD117, Stro-1, and HLA-DR.

Preferred methods include embodiments wherein said subepithelial umbilical cord derived mesenchymal stem cells are positive for SOX2.

Preferred methods include embodiments wherein said subepithelial umbilical cord derived mesenchymal stem cells are positive for OCT4.

Preferred methods include embodiments wherein said subepithelial umbilical cord derived mesenchymal stem cells are positive for OCT4 and SOX2.

Preferred methods include embodiments wherein said cells are directly injected into the uterine lining with or without ultrasound guidance.

Preferred methods include embodiments wherein said cells are directly injected into the uterine lining via transvaginal approach.

Preferred methods include embodiments wherein said cells are directly injected into the uterine lining via laparoscopic approach.

Preferred methods include embodiments wherein said cells are placed in carrier solution of less than 3 percent hematocrit platelet rich plasma for injection.

Preferred methods include embodiments wherein said cells are placed in carrier solution of reconstituted lyophilized or fresh platelet lysate for injection.

Preferred methods include embodiments wherein the carrier solution of less than 3 percent hematocrit platelet rich plasma or reconstituted lyophilized or fresh platelet lysate for injection is use for injection to prime the tissue.

### DETAILED DESCRIPTION OF THE INVENTION

For the practice of the invention, MSC are a type of stem cell utilized for inducing regeneration of the endometrium. “Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may include cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

In one embodiment, the cells of the present invention are generally referred to as umbilical-derived cells (or UDCs). They also may sometimes be referred to more generally herein as postpartum-derived cells or postpartum cells (PPDCs). In addition, the cells may be described as being stem or progenitor cells, the latter term being used in the broad sense. The term derived is used to indicate that the cells have been obtained from their biological source and grown or otherwise manipulated in vitro (e.g., cultured in a growth medium to expand the population and/or to produce a cell line). The in vitro manipulations of umbilical stem cells and the unique features of the umbilicus-derived cells of the present invention are described in detail below.

Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A conditioned medium is a medium in which a specific cell or population of cells has been cultured, and then removed. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. The medium containing the cellular factors is the conditioned medium. In one specific embodiment of the invention, supernatant is collected from MSC selected for ability to suppress fibrosis. In other embodiments, MSC are chosen based on angiogenic activity. Said angiogencic activity is identified based on proteomic and other analysis of markers, proteins, and peptides that are correlated with enhanced ability to induce regeneration. In a specific embodiment the invention provides means of regenerating endometrium using said conditioned media. In some embodiments of the invention, the inventors interchangeably use the words “conditioned media” and “trophic factors”. Generally, a trophic factor is defined as a substance that promotes or at least supports, survival, growth, proliferation and/or maturation of a cell, or stimulates increased activity of a cell.

When referring to cultured vertebrate cells, the term senescence (also replicative senescence or cellular senescence) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown, continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are actually resistant to programmed cell death (apoptosis), and have been maintained in their non-dividing state for as long as three years. These cells are very much alive and metabolically active, but they do not divide. The non-dividing state of senescent cells has not yet been found to be reversible by any biological, chemical, or viral agent.

As used herein, the term Growth Medium generally refers to a medium sufficient for the culturing of umbilicus-derived cells. In particular, one presently preferred medium for the culturing of the cells of the invention herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases, different growth media are used, or different supplementations are provided, and these are normally indicated in the text as supplementations to Growth Medium.

Also relating to the present invention, the term standard growth conditions, as used herein refers to culturing of cells at 37 degrees C., in a standard atmosphere comprising 5% CO2. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO2, relative humidity, oxygen, growth medium, and the like.

In one embodiment MSC donor lots are generated from umbilical cord tissue. Means of generating umbilical cord tissue MSC have been previously published and are incorporated by reference [1-7]. The term “umbilical tissue derived cells (UTC)” refers, for example, to cells as described in U.S. Pat. Nos. 7,510,873, 7,413,734, 7,524,489, and 7,560,276. The UTC can be of any mammalian origin e.g. human, rat, primate, porcine and the like. In one embodiment of the invention, the UTC are derived from human umbilicus. umbilicus-derived cells, which relative to a human cell that is a fibroblast, a mesenchymal stem cell, or an iliac crest bone marrow cell, have reduced expression of genes for one or more of: short stature homeobox 2; heat shock 27 kDa protein 2; chemokine (C-X-C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeobox 2 (growth arrest-specific homeobox); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeobox 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E 1B 19 kDa interacting protein 3-like; AE binding protein 1; and cytochrome c oxidase subunit VIIa polypeptide 1 (muscle). In addition, these isolated human umbilicus-derived cells express a gene for each of interleukin 8; reticulon 1; chemokine (C-X-C motif) ligand 1 (melonoma growth stimulating activity, alpha); chemokine (C-X-C motif) ligand 6 (granulocyte chemotactic protein 2); chemokine (C-X-C motif) ligand 3; and tumor necrosis factor, alpha-induced protein 3, wherein the expression is increased relative to that of a human cell which is a fibroblast, a mesenchymal stem cell, an iliac crest bone marrow cell, or placenta-derived cell. The cells are capable of self-renewal and expansion in culture, and have the potential to differentiate into cells of other phenotypes.

Methods of deriving cord tissue mesenchymal stem cells from human umbilical tissue are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes. Tissue can be obtained from any completed pregnancy, term or less than term, whether delivered vaginally, or through other routes, for example surgical Cesarean section. Obtaining tissue from tissue banks is also considered within the scope of the present invention.

The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the umbilical tissues can be accomplished by any of the various techniques known in the art, including by mechanical disruption. For example, tissue can be aseptically cut with scissors, or a scalpel, or such tissue can be otherwise minced, blended, ground, or homogenized in any manner that is compatible with recovering intact or viable cells from human tissue.

In a presently preferred embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatible herewith includes mucolytic enzyme activities, metalloproteases, neutral proteases, serine proteases (such as trypsin, chymotrypsin, or elastase), and deoxyribonucleases. Presently preferred are enzyme activities selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37 degree C. during the enzyme treatment of the dissociation step. Diluting the digest may also improve yields of cells as cells may be trapped within a viscous digest.

While the use of enzyme activities is presently preferred, it is not required for isolation methods as provided herein. Methods based on mechanical separation alone may be successful in isolating the instant cells from the umbilicus as discussed above or using TryplE.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells.

Providing the growth conditions allows for a wide range of options as to culture medium, supplements, atmospheric conditions, and relative humidity for the cells. A preferred temperature is 37 degrees C., however the temperature may range from about 35 degrees C. to 39 degrees C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above; however, they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, cord tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR, DP, DQ.

In one embodiment, bone marrow MSC lots are generated, means of generating BM MSC are known in the literature and examples are incorporated by reference.

In one embodiment BM-MSC are generated as follows:
1. 500 mL Isolation Buffer is prepared (PBS+2% FBS+2 mM EDTA) using sterile components or filtering Isolation Buffer through a 0.2 micron filter. Once made, the Isolation Buffer was stored at 2-8.degree. C.
2. The total number of nucleated cells in the BM sample is counted by taking 10.mu.L BM and diluting it 1/50-1/100 with 3% Acetic Acid with Methylene Blue (STEMCELL Catalog #07060). Cells are counted using a hemacytometer.
3. 50 mL Isolation Buffer is warmed to room temperature for 20 minutes prior to use and bone marrow was diluted 5/14 final dilution with room temperature Isolation Buffer (e.g. 25 mL BM was diluted with 45 mL Isolation Buffer for a total volume of 70 mL).
4. In three 50 mL conical tubes (BD Catalog #352070), 17 mL Ficoll-Paque™ PLUS (Catalog #07907/07957) is pipetted into each tube. About 23 mL of the diluted BM from step 3 was carefully layered on top of the Ficoll-Paque™ PLUS in each tube.
5. The tubes are centrifuged at room temperature (15-25.degree. C.) for 30 minutes at 300.times.g in a bench top centrifuge with the brake off.
6. The upper plasma layer is removed and discarded without disturbing the plasma:Ficoll-Paque™ PLUS interface. The mononuclear cells located at the interface layer are carefully removed and placed in a new 50 mL conical tube. Mononuclear cells are resuspended with 40 mL cold (2-8.degree. C.) Isolation Buffer and mixed gently by pipetting.
7. Cells were centrifuged at 300.times.g for 10 minutes at room temperature in a bench top centrifuge with the brake on. The supernatant is removed and the cell pellet resuspended in 1-2 mL cold Isolation Buffer.
8. Cells were diluted 1/50 in 3% Acetic Acid with Methylene Blue and the total number of nucleated cells counted using a hemacytometer.
9. Cells are diluted in Complete Human MesenCult®-Proliferation medium (STEMCELL catalog #05411) at a final concentration of 1.times.10.sup.6 cells/mL.
10. BM-derived cells were ready for expansion and CFU-F assays in the presence of GW2580, which can then be used for specific applications.

In one embodiment, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2′10 7 cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1′10 6 cells per ml in 175 cm 2 tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1′10⁶ per 175 cm². Said bone marrow MSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

Cell cultures are tested for sterility weekly, endotoxin by limulus amebocyte lysate test, and mycoplasma by DNA-fluorochrome stain.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody. Confluent MSC in 175 cm 2 flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington DC, USA). MSCs harvested from each 10-flask set were stored for up to 4 h at 4° C. and combined at the end of the harvest. A total of 2-10′10⁶ MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, IL, USA) freezing bags using a rate controlled freezer at a final concentration of 10% DMSO (Research Industries, Salt Lake City, UT, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

In one embodiment of the invention MSC to be used for treatment of endometrial regeneration are transfected with anti-apoptotic proteins to enhance in vivo longevity. The present invention includes a method of using MSC that have been cultured under conditions to express increased amounts of at least one anti-apoptotic protein as a therapy to inhibit or prevent apoptosis. In one embodiment, the MSC which are used as a therapy to inhibit or prevent apoptosis have been contacted with an apoptotic cell. The invention is based on the discovery that MSC that have been contacted with an apoptotic cell express high levels of anti-apoptotic molecules. In some instances, the MSC that have been contacted with an apoptotic cell secrete high levels of at least one anti-apoptotic protein, including but not limited to, STC-1, BCL-2, XIAP, Survivin, and Bcl-2XL. Methods of transfecting antiapoptotic genes into MSC have been previously described which can be applied to the current invention, said antiapoptotic genes that can be utilized for practice of the invention, in a nonlimiting way, include GATA-4 [8], FGF-2 [9], bcl-2 [10, 11], and HO-1 [12]. Based upon the disclosure provided herein, MSC can be obtained from any source. The MSC may be autologous with respect to the recipient (obtained from the same host) or allogeneic with respect to the recipient. In addition, the MSC may be xenogeneic to the recipient (obtained from an animal of a different species). In one embodiment of the invention MSC are pretreated with agents to induce expression of antiapoptotic genes, one example is pretreatment with exendin-4 as previously described [13]. In a further non-limiting embodiment, MSC used in the present invention can be isolated, from the bone marrow of any species of mammal, including but not limited to, human, mouse, rat, ape, gibbon, bovine. In a non-limiting embodiment, the MSC are isolated from a human, a mouse, or a rat. In another non-limiting embodiment, the MSC are isolated from a human.

Based upon the present disclosure, MSC can be isolated and expanded in culture in vitro to obtain sufficient numbers of cells for use in the methods described herein provided that the MSC are cultured in a manner that promotes contact with a tumor endothelial cell. For example, MSC can be isolated from human bone marrow and cultured in complete medium (DMEM low glucose containing 4 mM L-glutamine, 10% FBS, and 1% penicillin/streptomycin) in hanging drops or on non-adherent dishes. The invention, however, should in no way be construed to be limited to any one method of isolating and/or to any culturing medium. Rather, any method of isolating and any culturing medium should be construed to be included in the present invention provided that the MSC are cultured in a manner that provides MSC to express increased amounts of at least one anti-apoptotic protein. Culture conditions for growth of clinical grade MSC have been described in the literature and are incorporated by reference [14-47].

Cell cultures are tested for sterility weekly, endotoxin by limulus amebocyte lysate test, and mycoplasma by DNA-fluorochrome stain.

For the practice of the in invention MSC may be purified from various cellular sources such as bone marrow cells [48-54], umbilical cord tissue [55-57], peripheral blood [58-60], amniotic membrane [61], amniotic fluid, mobilized peripheral blood [62], adipose tissue [63, 64], endometrium and other tissues. When tissue sources of MSC are used said tissue isolates from which the MSC are isolated comprise a mixed populations of cells. MSC with endometrial stimulating activity constitute a very small percentage in these initial populations. They must be purified away from the other cells before they can be expanded in culture sufficiently to obtain enough cells for therapeutic applications.

Examples of compositions comprising endometrial promoting MSC include liquid preparations, including suspensions and preparations for intramuscular or intravenous administration (e.g., injectable administration), such as sterile suspensions or emulsions. Such compositions may comprise an admixture of endometrial promoting MSC with a suitable carrier, diluent, or excipient such as sterile water, physiological saline, glucose, dextrose, or the like. The compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE,” 17th edition, 1985, incorporated herein by reference, may be consulted to prepare suitable preparations, without undue experimentation.

Compositions of the invention often are conveniently provided as liquid preparations, e.g., isotonic aqueous solutions, suspensions, emulsions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues.

Various additives often will be included to enhance the stability, sterility, and isotonicity of the compositions, such as antimicrobial preservatives, antioxidants, chelating agents, and buffers, among others. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents that delay absorption, for example, aluminum monostearate, and gelatin. According to the present invention, however, any vehicle, diluent, or additive used would have to be compatible with the cells.

Endometrial promoting MSC solutions, suspensions, and gels normally contain a major amount of water (preferably purified, sterilized water) in addition to the cells. Minor amounts of other ingredients such as pH adjusters (e.g., a base such as NaOH), emulsifiers or dispersing agents, buffering agents, preservatives, wetting agents and jelling agents (e.g., methylcellulose) may also be present.

Typically, the compositions will be isotonic, i.e., they will have the same osmotic pressure as blood and lacrimal fluid when properly prepared for administration.

The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount, which will achieve the selected viscosity. Viscous compositions are normally prepared from solutions by the addition of such thickening agents.

A pharmaceutically acceptable preservative or cell stabilizer can be employed to increase the life of endometrial stimulating MSC compositions. If such preservatives are included, it is well within the purview of the skilled artisan to select compositions that will not affect the viability or efficacy of the endometrial stimulating MSC.

Those skilled in the art will recognize that the components of the compositions should be chemically inert. This will present no problem to those skilled in chemical and pharmaceutical principles. Problems can be readily avoided by reference to standard texts or by simple experiments (not involving undue experimentation) using information provided by the disclosure, the documents cited herein, and generally available in the art.

Sterile injectable solutions can be prepared by incorporating the cells utilized in practicing the present invention in the required amount of the appropriate solvent with various amounts of the other ingredients, as desired.

In some embodiments, endometrial promoting MSC are formulated in a unit dosage injectable form, such as a solution, suspension, or emulsion. Pharmaceutical formulations suitable for injection of endometrial promoting MSC typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), platelet containing plasma with low hematocrit to prevent toxicity, platelet lysate, either fresh or reconstituted lyophilized platelet lysate to buffer the cell product and suitable mixtures thereof.

The skilled artisan can readily determine the number of cells and optional additives, vehicles, and/or carrier in compositions to be administered in methods of the invention. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

To increase retention and survival of the cells a pretreatment of the uterine and/or ovarian tissue may be required using the carrier solution of platelet containing plasma with low hematocrit or, platelet lysate, either fresh or reconstituted lyophilized platelet lysate.

For any composition to be administered to an animal or human, and for any particular method of administration, it is preferred to determine therefore: toxicity, such as by determining the lethal dose (LD) and LD50 in a suitable animal model, e.g., rodent such as mouse or rat; and, the dosage of the composition(s), concentration of components therein, and timing of administering the composition(s), which elicit a suitable response. Such determinations do not require undue experimentation from the knowledge of the skilled artisan, this disclosure, and the documents cited herein. And, the time for sequential administrations can be ascertained without undue experimentation.

In various embodiments of the invention endometrial promoting MSC are administered by systemic injection. Systemic injection, such as intravenous injection, offers one of the simplest and least invasive routes for administering endometrial promoting MSC. In some cases, these routes may require high endometrial promoting MSC doses for optimal effectiveness and/or homing by the endometrial promoting MSC to the target sites. In a variety of embodiments endometrial promoting MSC may be administered by targeted and/or localized injections to ensure optimum effect at the target sites.

Endometrial promoting MSC may be administered to the subject through a hypodermic needle by a syringe in some embodiments of the invention. In various embodiments, endometrial promoting MSC are administered to the subject through a catheter. In a variety of embodiments, endometrial promoting MSC are administered by surgical implantation. Further in this regard, in various embodiments of the invention, endometrial promoting MSC are administered to the subject by implantation using a laparoscopic procedure. In some embodiments endometrial promoting MSC are administered to the subject in or on a solid support, such as a polymer or gel. In various embodiments, endometrial promoting MSC are administered to the subject in an encapsulated form.

In additional embodiments of the invention, endometrial promoting MSC are suitably formulated for oral, rectal, percutaneous, ocular, nasal, and/or pulmonary delivery and are administered accordingly.

Compositions can be administered in dosages and by techniques well known to those skilled in the medical and veterinary arts taking into consideration such factors as the age, sex, weight, and condition of the particular patient, and the formulation that will be administered (e.g., solid vs. liquid). Doses for humans or other mammals can be determined without undue experimentation by the skilled artisan, from this disclosure, the documents cited herein, and the knowledge in the art.

The dose of endometrial promoting MSC appropriate to be used in accordance with various embodiments of the invention will depend on numerous factors. It may vary considerably for different circumstances. The parameters that will determine optimal doses of endometrial promoting MSC to be administered for primary and adjunctive therapy generally will include some or all of the following: the disease being treated and its stage; the species of the subject, their health, gender, age, weight, and metabolic rate; the subject's immunocompetence; other therapies being administered; and expected potential complications from the subject's history or genotype. The parameters may also include: whether the endometrial promoting MSC are syngeneic, autologous, allogeneic, or xenogeneic; their potency (specific activity); the site and/or distribution that must be targeted for the endometrial promoting MSC to be effective; and such characteristics of the site such as accessibility to endometrial promoting MSC and/or engraftment of endometrial promoting MSC. Additional parameters include co-administration with endometrial promoting MSC of other factors (such as growth factors and cytokines). The optimal dose in a given situation also will take into consideration the way in which the cells are formulated, the way they are administered, and the degree to which the cells will be localized at the target sites following administration. Finally, the determination of optimal dosing necessarily will provide an effective dose that is neither below the threshold of maximal beneficial effect nor above the threshold where the deleterious effects associated with the dose of endometrial promoting MSC outweighs the advantages of the increased dose.

The optimal dose of endometrial promoting MSC for some embodiments will be in the range of doses used for autologous, mononuclear bone marrow transplantation. For fairly pure preparations of endometrial promoting MSC, optimal doses in various embodiments will range from 10.sup.4 to 10.sup.8 endometrial promoting MSC cells/kg of recipient mass per administration. In some embodiments the optimal dose per administration will be between 10.sup.5 to 10.sup.7 endometrial promoting MSC cells/kg. In many embodiments the optimal dose per administration will be 5.times.10.sup.5 to 5.times.10.sup.6 endometrial promoting MSC cells/kg. By way of reference, higher doses in the foregoing are analogous to the doses of nucleated cells used in autologous mononuclear bone marrow transplantation. Some of the lower doses are analogous to the number of CD34.sup.+ cells/kg used in autologous mononuclear bone marrow transplantation.

It is to be appreciated that a single dose may be delivered all at once, fractionally, or continuously over a period of time. The entire dose also may be delivered to a single location or spread fractionally over several locations.

In various embodiments, endometrial promoting MSC may be administered in an initial dose, and thereafter maintained by further administration of endometrial promoting MSC. Endometrial promoting MSC may be administered by one method initially, and thereafter administered by the same method or one or more different methods. The subject's MSC levels can be maintained by the ongoing administration of the cells. Various embodiments administer the Enhanced MSC either initially or to maintain their level in the subject or both by intravenous injection. In a variety of embodiments, other forms of administration, are used, dependent upon the patient's condition and other factors, discussed elsewhere herein.

It is noted that human subjects are treated generally longer than experimental animals; but, treatment generally has a length proportional to the length of the disease process and the effectiveness of the treatment. Those skilled in the art will take this into account in using the results of other procedures carried out in humans and/or in animals, such as rats, mice, non-human primates, and the like, to determine appropriate doses for humans. Such determinations, based on these considerations and taking into account guidance provided by the present disclosure and the prior art will enable the skilled artisan to do so without undue experimentation.

Suitable regimens for initial administration and further doses or for sequential administrations may all be the same or may be variable. Appropriate regiments can be ascertained by the skilled artisan, from this disclosure, the documents cited herein, and the knowledge in the art.

The dose, frequency, and duration of treatment will depend on many factors, including the nature of the disease, the subject, and other therapies that may be administered. Accordingly, a wide variety of regimens may be used to administer Enhanced MSC.

In some embodiments Enhanced MSC are administered to a subject in one dose. In others Enhanced MSC are administered to a subject in a series of two or more doses in succession. In some other embodiments wherein Enhanced MSC are administered in a single dose, in two doses, and/or more than two doses, the doses may be the same or different, and they are administered with equal or with unequal intervals between them.

Enhanced MSC may be administered in many frequencies over a wide range of times. In some embodiments, enhanced MSC are administered over a period of less than one day. In other embodiment they are administered over two, three, four, five, or six days. In some embodiments Enhanced MSC are administered one or more times per week, over a period of weeks. In other embodiments they are administered over a period of weeks for one to several months. In various embodiments they may be administered over a period of months. In others they may be administered over a period of one or more years. Generally, lengths of treatment will be proportional to the length of the disease process, the effectiveness of the therapies being applied, and the condition and response of the subject being treated.

### REFERENCES

ADDIN EN.REFLIST 1. Van Pham, P., et al., Isolation and proliferation of umbilical cord tissue derived mesenchymal stem cells for clinical applications. Cell Tissue Bank, 2015.
2. Fazzina, R., et al., A new standardized clinical-grade protocol for banking human umbilical cord tissue cells. Transfusion, 2015. 55(12): p. 2864-73.
3. Bieback, K., Platelet lysate as replacement for fetal bovine serum in mesenchymal stromal cell cultures. Transfus Med Hemother, 2013. 40(5): p. 326-35.
4. Stanko, P., et al., Comparison of human mesenchymal stem cells derived from dental pulp, bone marrow, adipose tissue, and umbilical cord tissue by gene expression. Biomed Pap Med Fac Univ Palacky Olomouc Czech Repub, 2014. 158(3): p. 373-7.
5. Schira, J., et al., Significant clinical, neuropathological and behavioural recovery from acute spinal cord trauma by transplantation of a well-defined somatic stem cell from human umbilical cord blood. Brain, 2012. 135(Pt 2): p. 431-46.
6. Hartmann, I., et al., Umbilical cord tissue-derived mesenchymal stem cells grow best under GMP-compliant culture conditions and maintain their phenotypic and functional properties. J Immunol Methods, 2010. 363(1): p. 80-9.
7. Friedman, R., et al., Umbilical cord mesenchymal stem cells: adjuvants for human cell transplantation. Biol Blood Marrow Transplant, 2007. 13(12): p. 1477-86.
8. Yu, B., et al., Enhanced mesenchymal stem cell survival induced by GATA-4 overexpression is partially mediated by regulation of the miR-15 family. Int J Biochem Cell Biol, 2013. 45(12): p. 2724-35.
9. Xu, W., et al., Basic fibroblast growth factor expression is implicated in mesenchymal stem cells response to light-induced retinal injury. Cell Mol Neurobiol, 2013. 33(8): p. 1171-9.
10. Fang, Z., et al., Differentiation of GFP-Bcl-2-engineered mesenchymal stem cells towards a nucleus pulposus-like phenotype under hypoxia in vitro. Biochem Biophys Res Commun, 2013. 432(3): p. 444-50.
11. Li, W., et al., Bcl-2 engineered MSCs inhibited apoptosis and improved heart function. Stem Cells, 2007. 25(8): p. 2118-27.
12. Tsubokawa, T., et al., Impact of anti-apoptotic and anti-oxidative effects of bone marrow mesenchymal stem cells with transient overexpression of heme oxygenase-1 on myocardial ischemia. Am J Physiol Heart Circ Physiol, 2010. 298(5): p. H1320-9.
13. Zhou, H., et al., Exendin-4 protects adipose-derived mesenchymal stem cells from apoptosis induced by hydrogen peroxide through the PI3K/Akt-Sfrp2 pathways. Free Radic Biol Med, 2014. 77: p. 363-75.
14. Le Blanc, K., et al., Treatment of severe acute graft-versus-host disease with third party haploidentical mesenchymal stem cells. Lancet, 2004. 363(9419): p. 1439-41.
15. Lazarus, H. M., et al., Cotransplantation of HLA-identical sibling culture-expanded mesenchymal stem cells and hematopoietic stem cells in hematologic malignancy patients. Biol Blood Marrow Transplant, 2005. 11(5): p. 389-98.
16. Bernardo, M. E., et al., Optimization of in vitro expansion of human multipotent mesenchymal stromal cells for cell-therapy approaches: further insights in the search for a fetal calf serum substitute. J Cell Physiol, 2007. 211(1): p. 121-30.
17. Reinisch, A., et al., Humanized system to propagate cord blood-derived multipotent mesenchymal stromal cells for clinical application. Regen Med, 2007. 2(4): p. 371-82.
18. Capelli, C., et al., Human platelet lysate allows expansion and clinical grade production of mesenchymal stromal cells from small samples of bone marrow aspirates or marrow filter washouts. Bone Marrow Transplant, 2007. 40(8): p. 785-91.
19. Lataillade, J. J., et al., New approach to radiation burn treatment by dosimetry-guided surgery combined with autologous mesenchymal stem cell therapy. Regen Med, 2007. 2(5): p. 785-94.
20. Seshareddy, K., D. Troyer, and M. L. Weiss, Method to isolate mesenchymal-like cells from Wharton's Jelly of umbilical cord. Methods Cell Biol, 2008. 86: p. 101-19.
21. Sensebe, L., Clinical grade production of mesenchymal stem cells. Biomed Mater Eng, 2008. 18(1 Suppl): p. S3-10.
22. Sotiropoulou, P. A., S. A. Perez, and M. Papamichail, Clinical grade expansion of human bone marrow mesenchymal stem cells. Methods Mol Biol, 2007. 407: p. 245-63.
23. Shetty, P., et al., Clinical grade mesenchymal stem cells transdifferentiated under xenofree conditions alleviates motor deficiencies in a rat model of Parkinson's disease. Cell Biol Int, 2009. 33(8): p. 830-8.
24. Zhang, X., et al., Cotransplantation of HLA-identical mesenchymal stem cells and hematopoietic stem cells in Chinese patients with hematologic diseases. Int J Lab Hematol, 2010. 32(2): p. 256-64.
25. Arrigoni, E., et al., Isolation, characterization and osteogenic differentiation of adipose-derived stem cells: from small to large animal models. Cell Tissue Res, 2009. 338(3): p. 401-11.
26. Grisendi, G., et al., GMP-manufactured density gradient media for optimized mesenchymal stromal/stem cell isolation and expansion. Cytotherapy, 2010. 12(4): p. 466-77.
27. Prasad, V. K., et al., Efficacy and safety of ex vivo cultured adult human mesenchymal stem cells (Prochymal) in pediatric patients with severe refractory acute graft-versus-host disease in a compassionate use study. Biol Blood Marrow Transplant, 2011. 17(4): p. 534-41.
28. Sensebe, L., P. Bourin, and K. Tarte, Good manufacturing practices production of mesenchymal stem/stromal cells. Hum Gene Ther, 2011. 22(1): p. 19-26.
29. Capelli, C., et al., Minimally manipulated whole human umbilical cord is a rich source of clinical-grade human mesenchymal stromal cells expanded in human platelet lysate. Cytotherapy, 2011. 13(7): p. 786-801.
30. Ilic, N., et al., Manufacture of clinical grade human placenta-derived multipotent mesenchymal stromal cells. Methods Mol Biol, 2011. 698: p. 89-106.
31. Santos, F., et al., Toward a clinical-grade expansion of mesenchymal stem cells from human sources: a microcarrier-based culture system under xeno-free conditions. Tissue Eng Part C Methods, 2011. 17(12): p. 1201-10.
32. Timmins, N. E., et al., Closed system isolation and scalable expansion of human placental mesenchymal stem cells. Biotechnol Bioeng, 2012. 109(7): p. 1817-26.
33. Warnke, P. H., et al., A clinically feasible protocol for using human platelet lysate and mesenchymal stem cells in regenerative therapies. J Craniomaxillofac Surg, 2013. 41(2): p. 153-61.
34. Fekete, N., et al., GMP-compliant isolation and large-scale expansion of bone marrow-derived MSC. PLoS One, 2012. 7(8): p. e43255.
35. Hanley, P. J., et al., Manufacturing mesenchymal stromal cells for phase I clinical trials. Cytotherapy, 2013. 15(4): p. 416-22.
36. Trojahn Kolle, S. F., et al., Pooled human platelet lysate versus fetal bovine serum-investigating the proliferation rate, chromosome stability and angiogenic potential of human adipose tissue-derived stem cells intended for clinical use. Cytotherapy, 2013. p. 1086-97.
37. Veronesi, E., et al., Transportation conditions for prompt use of ex vivo expanded and freshly harvested clinical-grade bone marrow mesenchymal stromal/stem cells for bone regeneration. Tissue Eng Part C Methods, 2014. 20(3): p. 239-51.
38. Dolley-Sonneville, P. J., L. E. Romeo, and Z. K. Melkoumian, Synthetic surface for expansion of human mesenchymal stem cells in xeno-free, chemically defined culture conditions. PLoS One, 2013. 8(8): p. e70263.
39. Siciliano, C., et al., Optimization of the isolation and expansion method of human mediastinal-adipose tissue derived mesenchymal stem cells with virally inactivated GMP-grade platelet lysate. Cytotechnology, 2015. 67(1): p. 165-74.
40. Martins, J. P., et al., Towards an advanced therapy medicinal product based on mesenchymal stromal cells isolated from the umbilical cord tissue: quality and safety data. Stem Cell Res Ther, 2014. 5(1): p. 9.
41. Iudicone, P., et al., Pathogen-free, plasma poor platelet lysate and expansion of human mesenchymal stem cells. J Transl Med, 2014. 12: p. 28.
42. Skrahin, A., et al., Autologous mesenchymal stromal cell infusion as adjunct treatment in patients with multidrug and extensively drug-resistant tuberculosis: an open-label phase 1 safety trial. Lancet Respir Med, 2014. 2(2): p. 108-22.
43. Ikebe, C. and K. Suzuki, Mesenchymal stem cells for regenerative therapy: optimization of cell preparation protocols. Biomed Res Int, 2014. 2014: p. 951512.
44. Chatzistamatiou, T. K., et al., Optimizing isolation culture and freezing methods to preserve Wharton's jelly's mesenchymal stem cell (MSC) properties: an MSC banking protocol validation for the Hellenic Cord Blood Bank. Transfusion, 2014. 54(12): p. 3108-20.
45. Swamynathan, P., et al., Are serum free and xeno-free culture conditions ideal for large scale clinical grade expansion of Wharton's jelly derived mesenchymal stem cells? A comparative study. Stem Cell Res Ther, 2014. 5(4): p. 88.
46. Vaes, B., et al., Culturing protocols for human multipotent adult stem cells. Methods Mol Biol, 2015. 1235: p. 49-58.
47. Devito, L., et al., Wharton's jelly mesenchymal stromal/stem cells derived under chemically defined animal product free low oxygen conditions are rich in MSCA-1(+) subpopulation. Regen Med, 2014. 9(6): p. 723-32.
48. Nemunaitis, J., et al., Human marrow stromal cells: response to interleukin-6 (IL-6) and control of IL-6 expression. Blood, 1989. 74(6): p. 1929-35.
49. Sadovnikova, E. Y., et al., Induction of hematopoietic microenvironment by the extracellular matrix from long-term bone marrow cultures. Ann Hematol, 1991. 62(5): p. 160-4.
50. Lazarus, H. M., et al., Ex vivo expansion and subsequent infusion of human bone marrow-derived stromal progenitor cells (mesenchymal progenitor cells): implications for therapeutic use. Bone Marrow Transplant, 1995. 16(4): p. 557-64.
51. Yoo, J. U., et al., The chondrogenic potential of human bone-marrow-derived mesenchymal progenitor cells. J Bone Joint Surg Am, 1998. 80(12): p. 1745-57.
52. Fleming, J. E., Jr., et al., Monoclonal antibody against adult marrow-derived mesenchymal stem cells recognizes developing vasculature in embryonic human skin. Dev Dyn, 1998. 212(1): p. 119-32.
53. Ghilzon, R., C. A. McCulloch, and R. Zohar, Stromal mesenchymal progenitor cells. Leuk Lymphoma, 1999. 32(3-4): p. 211-21.
54. De Cesaris, V., et al., Isolation, proliferation and characterization of endometrial canine stem cells. Reprod Domest Anim, 2016.
55. Van Pham, P., et al., Isolation and proliferation of umbilical cord tissue derived mesenchymal stem cells for clinical applications. Cell Tissue Bank, 2016. 17(2): p. 289-302.
56. Zhao, G., et al., Large-scale expansion of Wharton's jelly-derived mesenchymal stem cells on gelatin microbeads, with retention of self-renewal and multipotency characteristics and the capacity for enhancing skin wound healing. Stem Cell Res Ther, 2015. 6: p. 38.
57. Huang, P., et al., Differentiation of human umbilical cord Wharton's jelly-derived mesenchymal stem cells into germ-like cells in vitro. J Cell Biochem, 2010. 109(4): p. 747-54.
58. Wang, S. J., et al., Chondrogenic Potential of Peripheral Blood Derived Mesenchymal Stem Cells Seeded on Demineralized Cancellous Bone Scaffolds. Sci Rep, 2016. 6: p. 36400.
59. Fazeli, Z., M. D. Omrani, and S. M. Ghaderian, CD29/CD184 expression analysis provides a signature for identification of neuronal like cells differentiated from PBMSCs. Neurosci Lett, 2016. 630: p. 189-93.
60. Wu, G., et al., Osteogenesis of peripheral blood mesenchymal stem cells in self assembling peptide nanofiber for healing critical size calvarial bony defect. Sci Rep, 2015. 5: p. 16681.
61. Shaer, A., et al., Isolation and characterization of Human Mesenchymal Stromal Cells Derived from Placental Decidua Basalis; Umbilical cord Wharton's Jelly and Amniotic Membrane. Pak J Med Sci, 2014. 30(5): p. 1022-6.
62. Fu, W. L., C. Y. Zhou, and J. K. Yu, A new source of mesenchymal stem cells for articular cartilage repair: MSCs derived from mobilized peripheral blood share similar biological characteristics in vitro and chondrogenesis in vivo as MSCs from bone marrow in a rabbit model. Am J Sports Med, 2014. 42(3): p. 592-601.
63. El-Badawy, A., et al., Adipose Stem Cells Display Higher Regenerative Capacities and More Adaptable Electro-Kinetic Properties Compared to Bone Marrow-Derived Mesenchymal Stromal Cells. Sci Rep, 2016. 6: p. 37801.
64. Plock, J. A., et al., The Influence of Timing and Frequency of Adipose-Derived Mesenchymal Stem Cell Therapy on Immunomodulation Outcomes After Vascularized Composite Allotransplantation. Transplantation, 2017. 101(1): p. el-ell.

1. A method of preventing and/or treating endometrial atrophy, comprising the steps of: a) selecting a patient in need of treatment; b) administering an effective number of regenerative cells into the subject in need of treatment; c) assessing effect of regenerative cell injecting/infusing and adjusting concentration and frequency based on response.

2. The method of claim 1, wherein said regenerative cells are administered into the ovarian and/or uterine artery.

3. The method of claim 2, wherein said regenerative cells are mesenchymal stem cell are derived from tissue comprising a group selected from: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) cord/peri-natal or placental tissue.

4. The method of claim 3, wherein said mesenchymal stem cells express a marker or plurality of markers selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

5. The method of claim 3, wherein said mesenchymal stem cells are generated from a pluripotent stem cell.

6. The method of claim 5, wherein said pluripotent stem cell is selected from a group comprising of: a) an embryonic stem cell; b) an inducible pluripotent stem cell; c) a parthenogenic stem cell; and d) a somatic cell nuclear transfer derived stem cell.

7. The method of claim 6, wherein said embryonic stem cell population expresses genes selected from a group comprising of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

8. The method of claim 6, wherein said inducible pluripotent stem cell possesses markers selected from a group comprising of: CD10, CD13, CD44, CD73, CD90, PDGFr-alpha, PD-L2, and HLA-A,B,C and possesses ability to undergo at least 40 doublings in culture, while maintaining a normal karyotype upon passaging.

9. The method of claim 6, wherein said parthenogenic stem cells wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.

10. The method of claim 5, wherein said mesenchymal stem cells are differentiated from a pluripotent stem cell source through culture in the presence of an inhibitor of the SMAD-2/3 pathway.

11. The method of claim 10, wherein said mesenchymal stem cells are differentiated from a pluripotent stem cell source through culture in the presence of an inhibitor nucleic acid targeting the SMAD-2/3 pathway.

12. The method of claim 11, wherein said nucleic acid inhibitor is selected from a group comprising of: a) an antisense oligonucleotide; b) a hairpin loop short interfering RNA; c) a chemically synthesized short interfering RNA molecule; and d) a hammerhead ribozyme.

13. The method of claim 12, wherein said small molecule inhibitor is SB-431542.

14. The method of 1, wherein the subject is known to Asherman's Syndrome.

15. The method of claim 14, wherein the subject has endometrial atrophy that is resistant to hormonal or other treatments.

16. The method of claim 15, wherein the subject has had one or more prior embryo implantation failures.

17. The method of claim 16, wherein the regenerative cells are prepared by administering to the subject an agent to mobilize regenerative cells from bone marrow into peripheral blood of the subject; and isolating said regenerative cells from peripheral blood of the subject.

18. The method of claim 17, wherein the agent to mobilize regenerative cells is IL-2.

19. The method of claim 17, wherein said regenerative cells are isolated with an antibody capable of binding cells expressing CD31 and CD34.

20. A method of preventing or treating endometrial atrophy comprising of administering regenerative cells combined with endometrial stimulating factors.
